(19) United States
(12) Patent Application Publication
(10) Pub. No.: US 2023/0093192 A1
(43) Pub. Date: Mar. 23, 2023

(54) SYSTEM, APPARATUS, AND METHOD FOR PROVIDING A TIP DURING A BROADCAST

(71) Applicant: HYTTO PTE. LTD., Singapore (SG)

(72) Inventors: Dan Liu, Guangzhou (CN); Jilin Qiu, Guangzhou (CN)

(21) Appl. No.: 17/934,290

(22) Filed: Sep. 22, 2022

Related U.S. Application Data
(63) Continuation-in-part of application No. 17/579,839, filed on Jan. 20, 2022.
Application No. 17/579,839 is a continuation-in-part of application No. 17/717,917, filed on Apr. 11, 2022.
Application No. 17/717,917 is a continuation of application No. 16/352,876, filed on Mar. 14, 2019, now Pat. No. 11,311,453.
Application No. 16/352,876 is a continuation-in-part of application No. 16/835,808, filed on Mar. 31, 2020, now Pat. No. 11,452,669.
(60) Provisional application No. 62/830,195, filed on Apr. 5, 2019.

(51) Int. Cl. A61H 19/00; A61F 5/41

(52) U.S. Cl. CPC A61H 19/44; A61H 19/34; A61F 5/41; A61H 2201/5007; A61H 23/02

(57) ABSTRACT

A system is disclosed. The system has a tipping module, comprising computer-executable code stored in non-volatile memory, a processor, and a user device. The tipping module, the processor, and the user device are configured to display one or more multimedia content pages on a user interface of the user device, the one or more multimedia content pages associated with one or more live broadcast platforms, display one or more tipping graphical elements on the user interface of the user device, the one or more tipping graphical elements associated with the one or more multimedia content pages, and execute at least one tip operation on the one or more live broadcast platforms, the at least one tip operation associated with the one or more tipping graphical elements and responsive to a trigger instruction of any of the one or more tipping graphical elements.

### CROSS REFERENCE TO RELATED APPLICATIONS

This application is a continuation-in-part of U.S. patent application Ser. No. 17/579,839, filed Jan. 20, 2022. This application is also a continuation-in-part of U.S. patent application Ser. No. 17/717,917, filed Apr. 11, 2022, which is a continuation of U.S. patent application Ser. No. 16/352,876, filed Mar. 14, 2019 (now U.S. Pat. No. 11,311,453 issued on Apr. 26, 2022). This application is also a continuation-in-part of U.S. patent application Ser. No. 16/835,808, filed Mar. 31, 2020, which claims priority to U.S. Provisional Patent Application No. 62/830,195, filed Apr. 5, 2019. Each of the above applications is hereby incorporated by reference in its entirety.

### FIELD OF THE INVENTION

The present disclosure generally relates to a system, apparatus, and method for providing a tip, and more particularly to a system, apparatus, and method for providing a tip during a broadcast.

### BACKGROUND OF THE INVENTION

Conventional live broadcasting typically involves a streamer's device that streams broadcast content to multiple viewer devices. The viewers can enjoy watching a show broadcast by the streamer in real time and may interact with the streamer by sending tips, gifts, or messages. However, providing tips during a live broadcast using conventional systems is often burdensome. Conventional tipping systems are often inconvenient and distracting for users to utilize during a broadcast. Conventional tipping systems may cause a tipper to miss portions of a broadcast during tipping and accordingly not enjoy a broadcast as much as the tipper would otherwise have. Also, the burdens associated with conventional tipping methods can discourage potential tippers from tipping and reduce an overall amount of tips received.

For example, the process for sending tips using conventional systems are typically cumbersome, with viewers often repeating the same steps each time they send tips. During a broadcast, in order to interact with model users, viewers typically operate adult toys synchronously with tipping, which makes it inconvenient for viewers to tip. For example, because viewers cannot easily send tips to model while watching a broadcast, viewers may reduce the amount of tips they send because of the inconvenience of tipping.

Accordingly, a need in the art exists for an efficient and convenient technique for tipping during a live broadcast.

The exemplary disclosed system and method are directed to overcoming one or more of the shortcomings set forth above and/or other deficiencies in existing technology.

### SUMMARY OF THE INVENTION

In one exemplary aspect, the present disclosure is directed to a system. The system includes a tipping module, comprising computer-executable code stored in non-volatile memory, a processor, and a user device. The tipping module, the processor, and the user device are configured to display one or more multimedia content pages on a user interface of the user device, the one or more multimedia content pages associated with one or more live broadcast platforms, display one or more tipping graphical elements on the user interface of the user device, the one or more tipping graphical elements associated with the one or more multimedia content pages, and execute at least one tip operation on the one or more live broadcast platforms, the at least one tip operation associated with the one or more tipping graphical elements and responsive to a trigger instruction of any of the one or more tipping graphical elements.

In another aspect, the present disclosure is directed to a method. The system includes displaying one or more multimedia content pages on a user interface of a user device, the one or more multimedia content pages associated with one or more live broadcast platforms, displaying one or more tipping graphical elements on the user interface of the user device, the one or more tipping graphical elements associated with the one or more multimedia content pages, and executing at least one tip operation on the one or more live broadcast platforms, the at least one tip operation associated with the one or more tipping graphical elements and responsive to a trigger instruction of any of the one or more tipping graphical elements.

### BRIEF DESCRIPTION OF THE DRAWINGS

FIG. 1 is a schematic illustration of an exemplary system of the present invention;

FIG. 2 is a schematic illustration of an exemplary system of the present invention;

FIG. 3 is a schematic illustration of an exemplary system of the present invention;

FIG. 4 is a schematic illustration of an exemplary system of the present invention;

FIG. 5 is a schematic illustration of an exemplary system of the present invention;

FIG. 6 is a schematic illustration of an exemplary system of the present invention;

FIG. 7 is a schematic illustration of an exemplary system of the present invention;

FIG. 8 is a schematic illustration of an exemplary system of the present invention;

FIG. 9A is a schematic illustration of an exemplary system of the present invention;

FIG. 9B is a schematic illustration of an exemplary system of the present invention;

FIG. 10 is a schematic illustration of an exemplary system of the present invention;

FIG. 11 is a flowchart showing an exemplary process of the present invention;

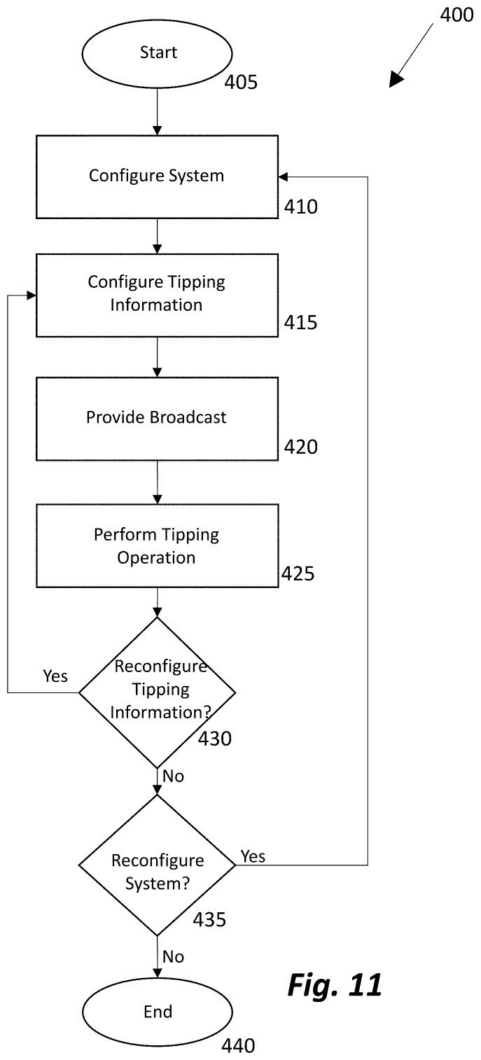

FIG. 12 is a schematic illustration of an exemplary computing device, in accordance with at least some exemplary embodiments of the present disclosure; and

FIG. 13 is a schematic illustration of an exemplary network, in accordance with at least some exemplary embodiments of the present disclosure.

### DETAILED DESCRIPTION AND INDUSTRIAL APPLICABILITY

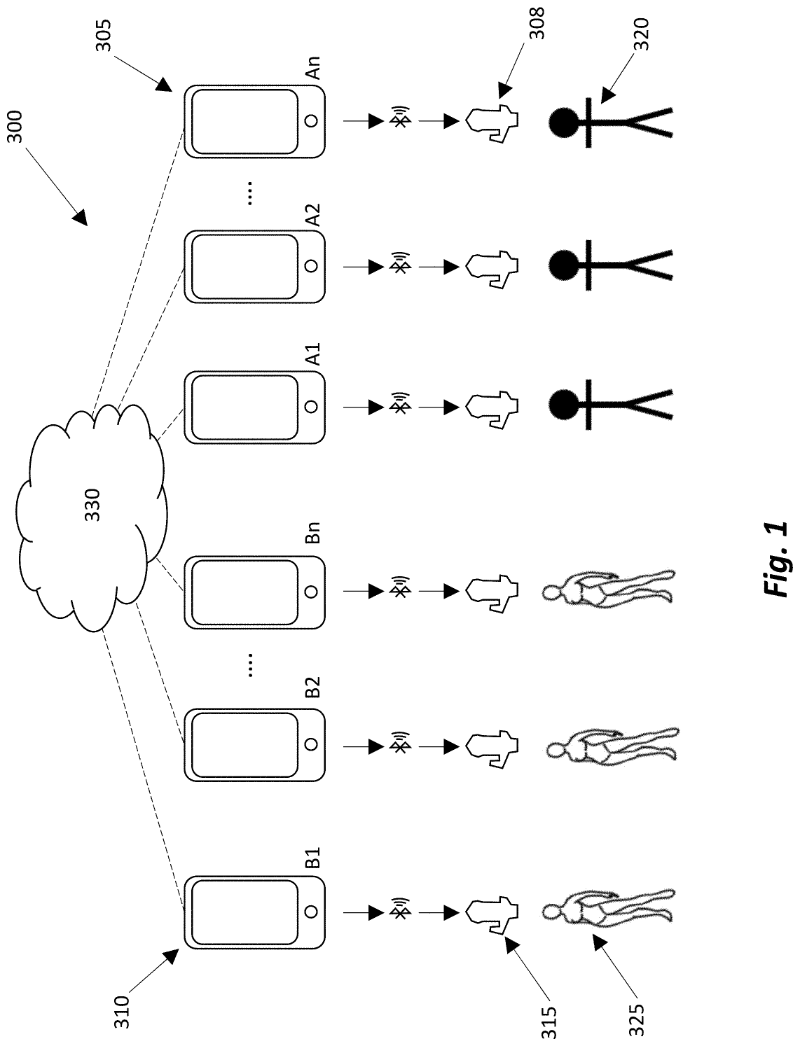

FIG. 1 illustrates an exemplary system 300 for synchronously controlling devices. In at least some exemplary embodiments, system 300 may be a system for providing a tip in real-time (e.g., in real-time or in near real-time) during a live broadcast of an adult entertainment application. The tip may be any suitable reward or unit of value (e.g., of value to a user). The tip may include (e.g., or represent and/or give access to) a national currency, a virtual currency, a commodity, stock, a credit for any desired use, and/or any other desired unit of value. The tip may include cryptocurrency and/or any other suitable currency.

System 300 may provide any desired tip such as a quick-tip or an auto-tip for example as described herein. System 300 may include any suitable imaging device such as a web camera and/or a camera of the exemplary disclosed user device. System 300 may provide a broadcast (e.g., a live broadcast or stream) between a streamer (e.g., a model) and a viewer that may be a tipper who provides tips. A model and/or a user may utilize an adult toy (e.g., a sex toy) for example as described herein.

As illustrated in FIG. 1, system 300 may include one or more user devices 305, one or more model devices 310, one or more viewer accessories 308, and/or one or more model accessories 315. For example, system 300 may include a plurality of user devices 305, a plurality of viewer accessories 308, a plurality of model devices 310, and/or a plurality of model accessories 315. Data such as image data, audio data, and/or control data may be transferred between user devices 305, viewer accessories 308, model devices 310, and model accessories 315.

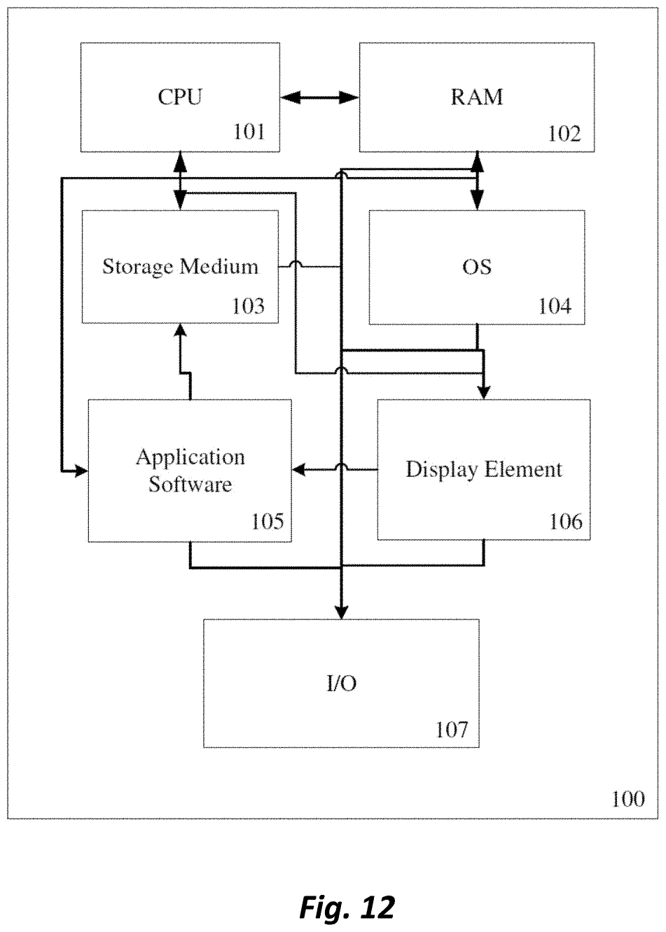

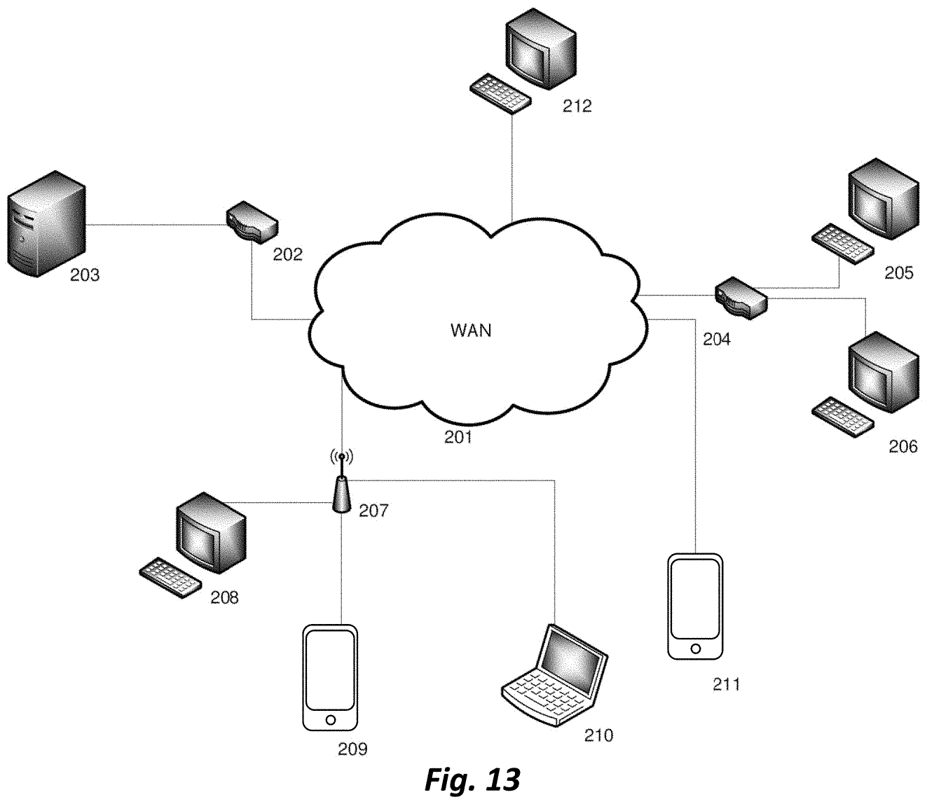

As illustrated in FIG. 1, system 300 may include any desired number of user devices 305 (e.g., A1, A2, . . . An). User device 305 may be any suitable device for interfacing with other components of system 300 such as a computing device (e.g., user interface). For example, user device 305 may be any suitable user interface for receiving input and/or providing output (e.g., image data) to a user 320. User device 305 may include a camera and a microphone. User device 305 may be, for example, a touchscreen device (e.g., of a smartphone, a tablet, a smartboard, and/or any suitable computer device), a wearable device, a computer keyboard and monitor (e.g., desktop or laptop), an audio-based device for entering input and/or receiving output via sound, a tactile-based device for entering input and receiving output based on touch or feel, a dedicated user interface designed to work specifically with other components of system 300, and/or any other suitable user interface (e.g., including components and/or configured to work with components described below regarding FIGS. 12 and 13). For example, user device 305 may include a touchscreen device of a smartphone or handheld tablet. For example, user device 305 may include a display (e.g., a computing device display, a touchscreen display, and/or any other suitable type of display) that may provide output, image data, and/or any other desired output or input prompt to a user. For example, the exemplary display may include a graphical user interface to facilitate entry of input by a user and/or receiving output such as image data. An application for example as described herein and/or a web browser may be installed on user device 305 and utilized by user 320.

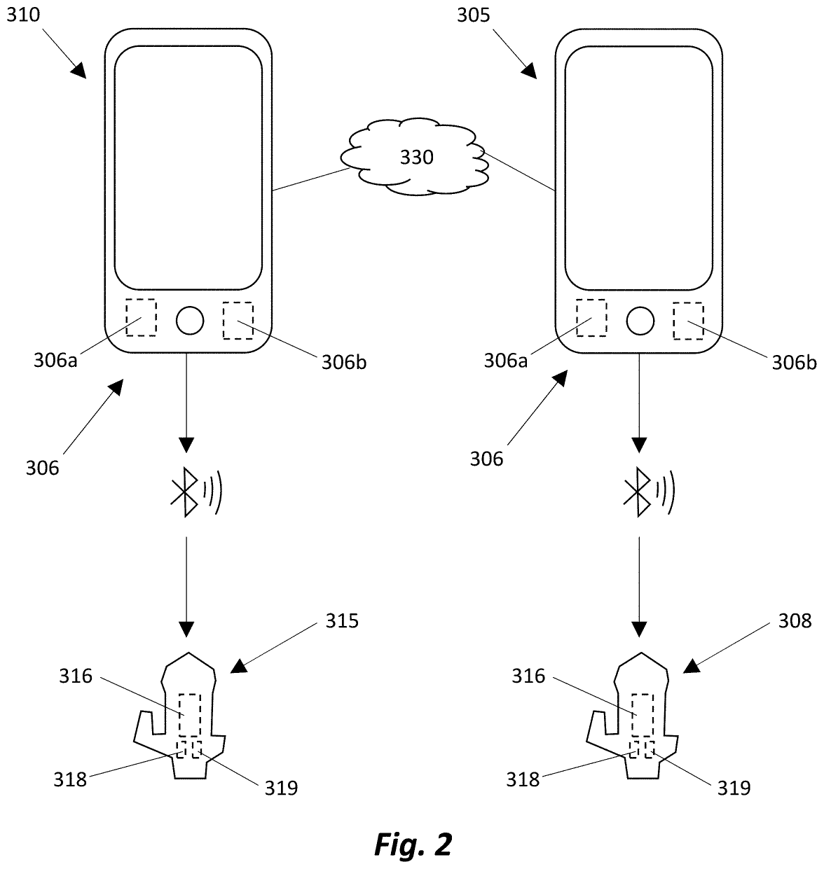

As illustrated in FIG. 2, user device 305 may include a sensor array 306. In at least some exemplary embodiments, sensor array 306 may include one or more sensors integrated or built into the exemplary disclosed user device (e.g., user device 305) such as, for example, a mobile phone, a pad, or a wearable device. Sensor array 306 may include any suitable sensors for use with system 300 such as, for example, a location sensor 306a and a movement sensor 306b. Location sensor 306a may include a GPS device, a Galileo device, a GLONASS device, an IRNSS device, a BeiDou device, and/or any other suitable device that may operate with a global navigation system.

Movement sensor 306b may include any suitable components for sensing motion (e.g., motion amplitude), velocity, and/or acceleration. Movement sensor 306b may include an acceleration sensor. Movement sensor 306b may include a gyroscope. For example, movement sensor 306b may include a displacement sensor, a velocity sensor, and/or an accelerometer. For example, movement sensor 306b may include components such as a servo accelerometer, a piezoelectric accelerometer, a potentiometric accelerometer, and/or a strain gauge accelerometer. Movement sensor 306b may include a piezoelectric velocity sensor or any other suitable type of velocity or acceleration sensor.

System 300 may include any desired number of model devices 310 (e.g., B1, B2, . . . Bn). Model device 310 may be similar to user device 305. For example, model device 310 may be any suitable user interface for receiving input and/or providing output (e.g., image data) to a model 325. Model 325 (e.g., a specific user) may operate model device 310 (e.g., a specific user device) to record and transfer image (e.g., video) and audio data to one or more users 320 via a network 330.

Model accessory 315 may be any suitable accessory for use by model 325 (e.g., when model 325 is imaged by model device 310). For example, model accessory 315 may be a prop that may be used by model 325 while model 325 is being imaged (e.g., a video or pictures of model 325 are being recorded and/or transmitted in real-time to be viewed by user 320). For example, model accessory 315 may be a device used for erotic stimulation (e.g., a sex aid or a “sex toy”). Model accessory 315 may be a sexual simulation device that may be associated with a given model 325 (e.g., a specific user) and respective model device 310 (e.g., a specific user device) of that given model 325. In at least some exemplary embodiments, model accessory 315 may be a massaging apparatus for human genitalia (e.g., a vibrator). For example, model accessory 315 may be any suitable device for use in a video or pictures recorded by model device 310, which may be an erotic video or erotic pictures. In at least some exemplary embodiments, model accessory 315 may be a tool or other indicator that may be used in video or pictures recorded by model device 310 such as surveying equipment, a sign providing information such as location or time information, a surveillance tool used by model 325, and/or any other suitable tool or accessory that may be used while model device 310 is recording a video or pictures of model 325. For example, model 325 may be an erotic model using model accessory 315 that may be an erotic device, a technician or laborer using model accessory 315 that may be a tool or work device specific to a desired application, an operative using model accessory 315 that may be a surveillance tool or a part of a weapon system being recorded by model device 310, and/or any other desired role using any suitable model accessory 315.

Model accessory 315 may include a motor 316. Motor 316 may include an electric motor. Motor 316 may include a server motor, a stepper motor, a brushless motor, or any other suitable type of motor. Motor 316 may include any suitable vibration motor or haptic motor such as, for example, a mini vibrator motor. Motor 316 may include a low voltage motor. Motor 316 may include a pager motor or a coin vibration motor. Motor 316 may include a linear resonant actuator or an eccentric rotating mass vibration motor. Motor 316 may be powered by any suitable power source, such as a battery (e.g., a nickel-metal hydride battery, a lithium-ion battery, an ultracapacitor battery, a lead-acid battery, and/or a nickel cadmium battery), an electric power source (e.g., a transformer connected to a plug that may plug into an outlet), and/or any other suitable energy source. Model accessory 315 may include a controller 319 that may be any suitable computing device for controlling an operation of motor 316 and a communication device 318. Controller 319 may, for example, include components similar to the components described below regarding FIG. 12. Controller 319 may include for example a processor (e.g., micro-processing logic control device) or board components. Controller 319 may control motor 316 based on input data and/or commands received from user device 305 and/or model device 310 via network 330 and/or a communication device 318 (e.g., transferred directly to communication device 318 by any suitable component of system 300). Motor 316 may be controlled by controller 319 to vibrate model accessory 315 at a desired level or strength, perform a suction operation at a desired level or strength using model accessory 315 (e.g., using model accessory 315 as a suction device), rotate or swing model accessory 315 at a desired speed or amount, contract or expand model accessory 315 by a desired amount, cause model accessory 315 to perform an inhalation action, and/or cause model accessory 315 to perform any other suitable action or function.

In at least some exemplary embodiments, motor 316 may be or may include a thermal device such as a heater. In at least some exemplary embodiments, motor 316 may include an electric heating device such as an electric resistance heating device. Motor 316 may include a polyimide heater, a silicone rubber heater, and/or a resistive wire heater. Motor 316 may be controlled by controller 319 to heat or emit heat or warmth from model accessory 315. For example, motor 316 may cause a temperature variation of model accessory 315.

Viewer accessory 308 may be similar to model accessory 315. Viewer accessory 308 may be a sexual simulation device that may be associated with a given user 320 (e.g., a viewer of one or more models 325) and respective user device 305 (e.g., a viewer device) of that given user 320.

Network 330 may be any suitable communication network over which data may be transferred between one or more user devices 305, one or more viewer accessories 308, one or more model devices 310, and/or one or more model accessories 315. Network 330 may be the internet, a LAN (e.g., via Ethernet LAN), a WAN, a WiFi network, or any other suitable network. Network 330 may be similar to WAN 201 described below. The components of system 300 may also be directly connected (e.g., by wire, cable, USB connection, and/or any other suitable electro-mechanical connection) to each other and/or connected via network 330. For example, components of system 300 may wirelessly transmit data by any suitable technique such as, e.g., wirelessly transmitting data via 4G LTE networks (e.g., or 5G networks) or any other suitable data transmission technique for example via network communication. Components of system 300 may transfer data via the exemplary techniques described below regarding FIG. 13. User devices 305, viewer accessories 308, model devices 310, and/or model accessories 315 may include any suitable communication components for communicating with other components of system 300 using for example the communication techniques described above. For example, user devices 305 and model devices 310 may include integrally formed communication devices (e.g., smartphone components), and viewer accessories 308 and model accessories 315 may each include communication device 318 that may communicate using any of the exemplary disclosed communication techniques.

In at least some exemplary embodiments, a given model accessory 315 may communicate with a given model device 310 (e.g., a paired model device 310) via any suitable short distance communication technique. For example, model accessories 315 (e.g., via communication device 318) and model devices 310 may communicate via WiFi, Bluetooth, ZigBee, NFC, IrDA, and/or any other suitable short distance technique. Model accessory 315 may be an adult toy that may be connected with model device 310 through short distance wireless communication. An application (e.g., operating using the exemplary disclosed modules) may be installed on model device 310, the application and model device 310 being configured to send commands to model accessory 315 to drive (e.g., actuate) model accessory 315. Viewer accessory 308 may communicate with user device 305 similarly to the communication of model accessory 315 and model device 310 described above.

System 300 may include one or modules for performing the exemplary disclosed operations. The one or more modules may include an accessory control module for controlling viewer accessory 308 and model accessory 315. The one or more modules may be stored and operated by any suitable components of system 300 (e.g., including processor components) such as, for example, network 330, user device 305, viewer accessory 308, model device 310, model accessory 315, and/or any other suitable component of system 300. For example, system 300 may include one or more modules having computer-executable code stored in non-volatile memory. System 300 may also include one or more storages (e.g., buffer storages) that may include components similar to the exemplary disclosed computing device and network components described below regarding FIGS. 12 and 13. For example, the exemplary disclosed buffer storage may include components similar to the exemplary storage medium and RAM described below regarding FIG. 12. The exemplary disclosed buffer storage may be implemented in software and/or a fixed memory location in hardware of system 300. The exemplary disclosed buffer storage (e.g., a data buffer) may store data temporarily during an operation of system 300.

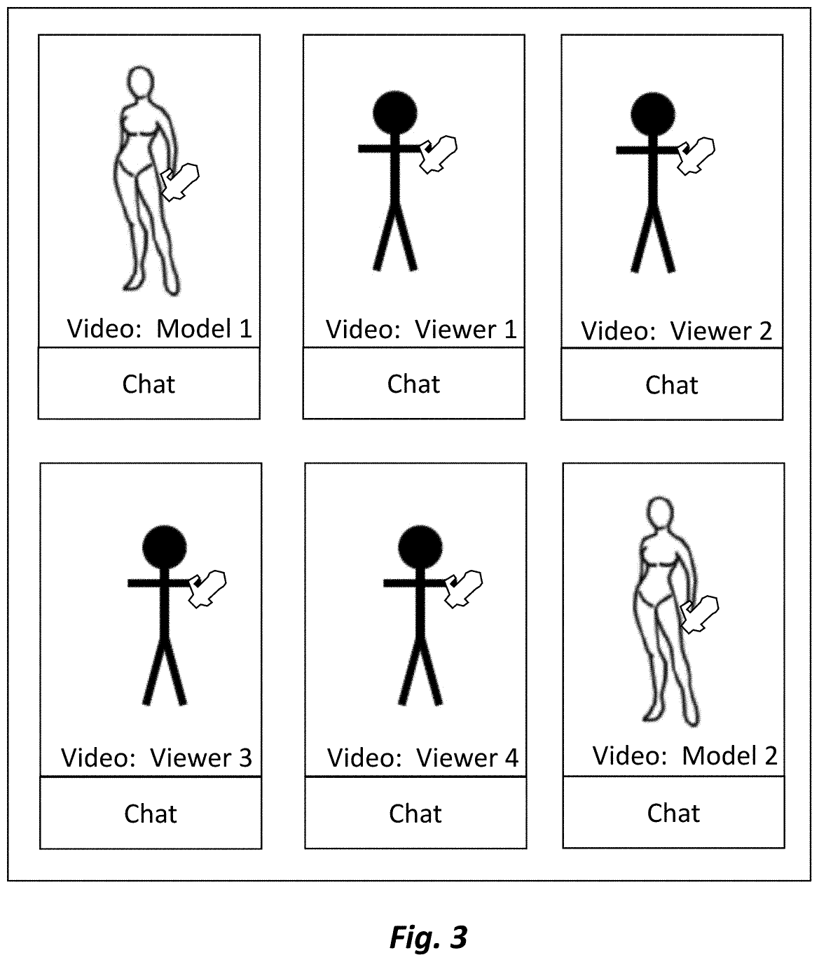

The one or more exemplary disclosed modules may also provide a chat room interface via user device 305 and model device 310 for use by each user 320 and model 325. For example, video display of model 325, one or more users 320, and/or and a chat or messaging app (e.g., any suitable chat communication or messaging app such as, for example, text, voice, and/or video chat boxes) may be displayed to each user 320 via user device 305 and to each model 325 via model device 310. One or more users 320 and one or more models 325 may thereby view and chat (e.g., text, voice, and/or video chat) with each other via the one or more exemplary disclosed modules via respective user devices 305 and model devices 310. Each user 320 may thereby view, interact with, and/or chat (e.g., text, voice, and/or video chat) with one or more models 325 and/or other users 320. Also, each model 325 may thereby view, interact with, and/or chat with one or users 320 and/or other models 325. For example, multiple text, voice, and/or video chat boxes including a plurality of users 320 (e.g., viewers each having one or more viewer accessories 308) and/or a plurality of models 325 (e.g., each having one or more model accessories 315) may be displayed to each user 320 and each model 325 via respective user devices 305 and model devices 310. Users 320 and models 325 may thereby view and interact with other users 320 and models 325 that may each have one or more respective accessories (e.g., respective viewer accessories 308 and model accessories 315). FIG. 3 schematically illustrates an exemplary embodiment of the exemplary disclosed chat room that may be displayed to user 320 via user device 305 or to model 325 via model device 310.

The exemplary disclosed system, apparatus, and method may be used in any suitable telecommunications application. The exemplary disclosed system, apparatus, and method may be used in any suitable application for providing a live broadcast. The exemplary disclosed system, apparatus, and method may be used in any suitable application for providing entertainment based on viewing images or videos. The exemplary disclosed system, apparatus, and method may be used in any suitable application for providing a tip. For example, the exemplary disclosed system, apparatus, and method may be used in any suitable application for providing a tip during a live broadcast. The exemplary disclosed system, apparatus, and method may be used in any suitable telecommunication application for adult entertainment.

In at least some exemplary embodiments, users (e.g., one or more users 320 and/or models 325) may access a live broadcast platform website (e.g., an application associated with the exemplary disclosed module) via the exemplary disclosed user device (e.g., via a browser on a device). The exemplary disclosed live broadcast platform website (e.g., as illustrated for example in FIGS. 3-10) may provide multiple multimedia pages for users to utilize (e.g., a live broadcast room page). For example, a live broadcast room may be used to allow two or more users (e.g., including viewers and models such as one or more users 320 and one or more models 325) to communicate. After a viewer (e.g., user 320) enters a live broadcast room using the exemplary disclosed application, the viewer may watch a performance and/or establish communication with one or more models (e.g., models 325) though the live broadcast room. The exemplary disclosed application (e.g., as illustrated for example in FIGS. 3-10) may also provide a live commenting screen, a chat window, and/or features for providing tips. For example, any suitable features may be provided on the exemplary disclosed live broadcast room page.

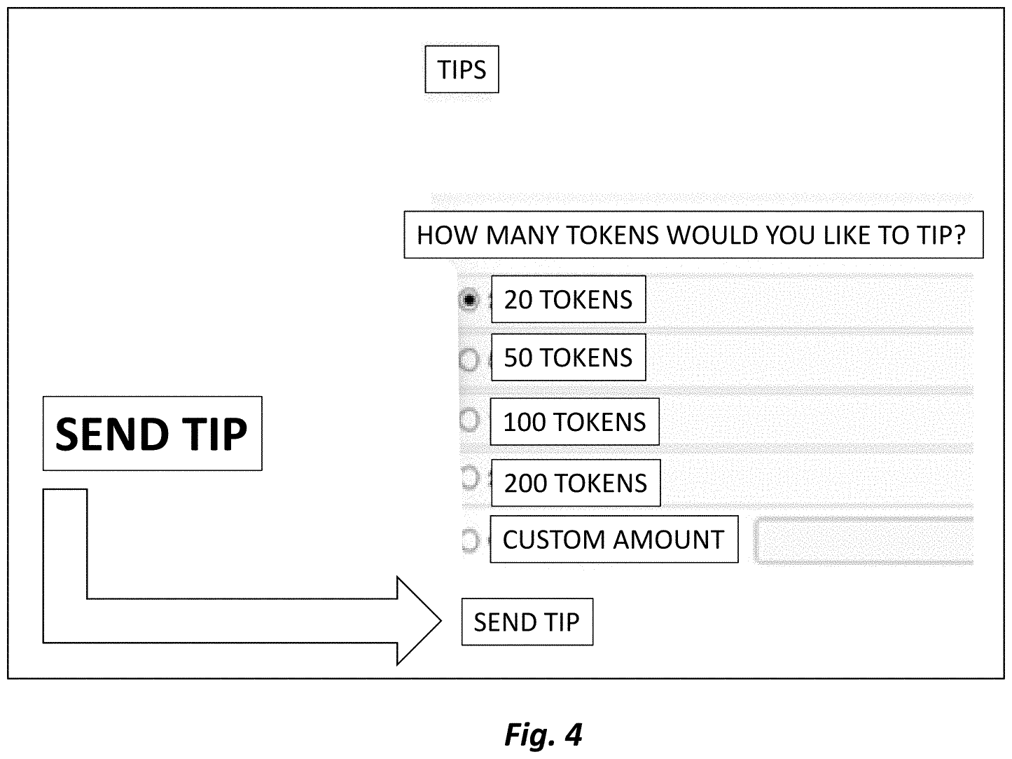

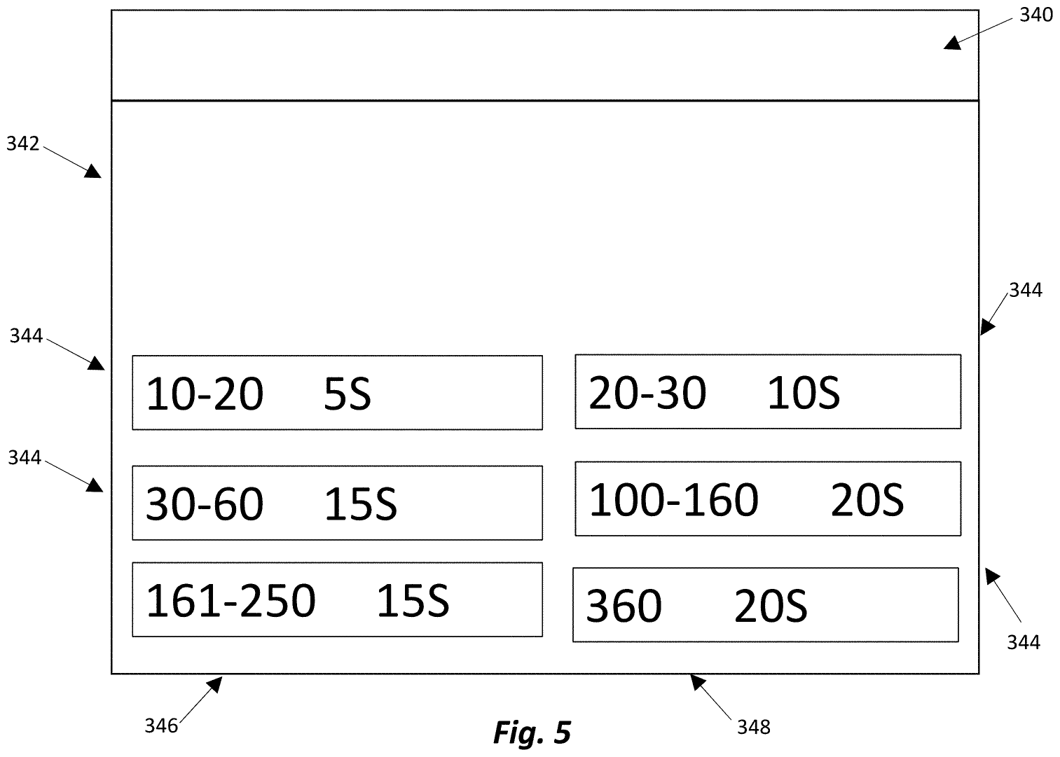

FIGS. 4 and 5 illustrate an exemplary disclosed graphical user interface (GUI) of system 300 that may be displayed using the exemplary disclosed user device (e.g., user device 305 and/or model device 310). FIGS. 4 and 5 illustrate an exemplary disclosed tip function for the exemplary disclosed GUI (e.g., providing a live broadcast platform). For example as illustrated in FIGS. 4 and 5, a user may select an amount of tokens to be provided to a model using the exemplary disclosed tipping process described herein.

In at least some exemplary embodiments, users such as viewers (e.g., users 320) may send tips via system 300 (e.g., to models 325) via the exemplary disclosed user devices and modules. For example, viewers may send tips after entering the exemplary disclosed live broadcast room (e.g., as illustrated for example in FIGS. 3-10).

For example as illustrated in FIG. 5, the exemplary disclosed application (e.g., live broadcasting platform) may include at least one multimedia content page. For example, the exemplary disclosed application may display two or more multimedia content pages (e.g., a plurality). The exemplary disclosed application may display a plurality of multimedia content pages simultaneously (e.g., at once). The multimedia content page may for example include a live broadcast room page, a video page, an audio page, an article page, a picture page, and/or any other suitable type of page (e.g., webpage). The exemplary disclosed GUI may include any suitable graphical elements (e.g., tipping items) that may be displayed on the exemplary disclosed multimedia content page (e.g., via the exemplary disclosed user interface).

In at least some exemplary embodiments and for example as illustrated in FIG. 5, the exemplary disclosed GUI may include a graphical element 340, a graphical element 342, a graphical element 344, a graphical element 346, and a graphical element 348. Graphical element 340 may be display and/or include multimedia content (e.g., a live broadcast room, a video, audio content, an article, a picture, and/or any other suitable type of content). In at least some exemplary embodiments, graphical element 342 may display a panel for example as illustrated in FIG. 5 (e.g., a panel displaying features of the exemplary disclosed quick-tip and/or auto-tip functions). Also for example, one or more tipping graphical elements 344 may be displayed on the exemplary disclosed user interface without graphical element 342.

Graphical elements 344 may include tipping items. For example, graphical elements 344 may include text, images, audio content, and/or any other suitable content including tipping information. The tipping information may include a duration (e.g., a duration of time in which an adult toy may be activated), a level of intensity of operation of a toy, an interval and/or pattern for activating the toy, and/or any other desired criteria for example as described herein. Tipping information of graphical elements 344 may be input by a user for example as described herein, predetermined, set by system 300 based on an algorithm or machine learning (e.g., based on past usage of the user), and/or any other desired criteria. For example as illustrated at graphical element 346, tipping information may include a range of values. Also for example as illustrated at graphical element 348, tipping information may include a fixed value. The exemplary disclosed values may display tipping ranges and/or amounts for example as illustrated in FIG. 5.

As illustrated in FIGS. 5-10, system 300 may provide quick-tip functions that may be integrated into the exemplary disclosed application (e.g., as plug-ins). The exemplary disclosed application may integrate a desired browser (e.g., a specified browser) or may be a browser application. The exemplary disclosed application may access the exemplary disclosed live broadcast platform website.

The exemplary disclosed quick-tip function may provide a tipping operation on a live broadcasting platform without a user performing multiple steps of tipping while utilizing the live platform. For example, system 300 may provide preset script and/or a preset interface associated with the application (e.g., a live broadcasting platform) for example as illustrated in FIGS. 5-10.

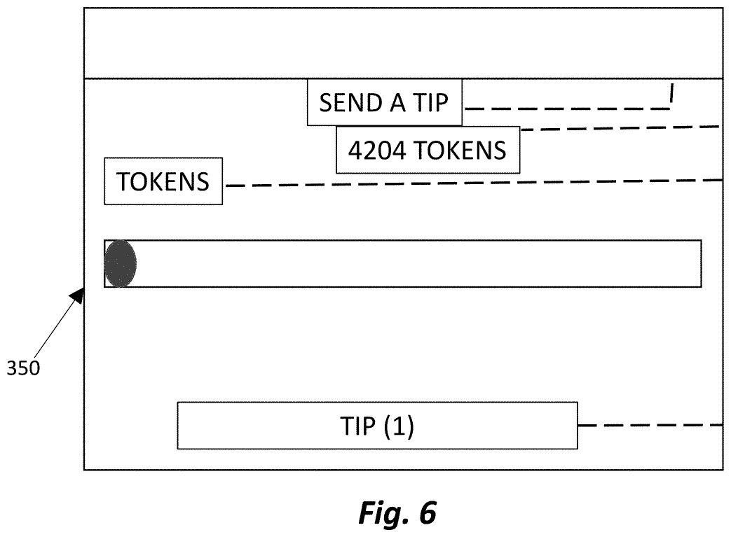

As illustrated in FIGS. 5 and 6, the exemplary disclosed graphical elements may include tipping information (e.g., including specified values or ranges of values of tips such as tip virtual currency). The values may include one or more different fixed values and/or one or more range of values for example as described herein regarding FIG. 5. Also for example as illustrated in FIG. 6, a user may select a target value from a range of values via the exemplary disclosed GUI. For example, a graphical element 350 may be utilized by a user to select a target value (e.g., selecting a fixed value or a range of values).

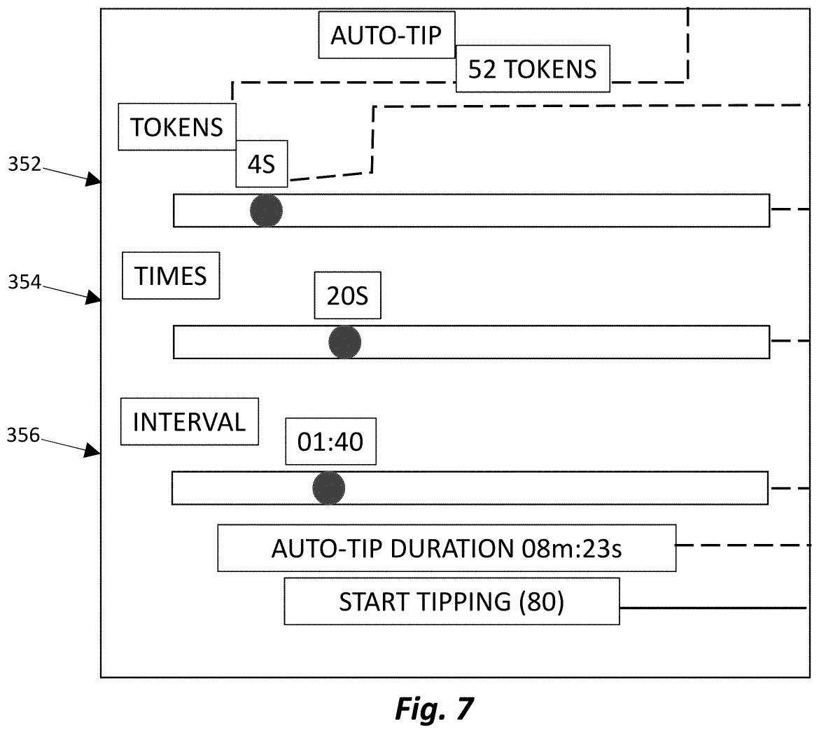

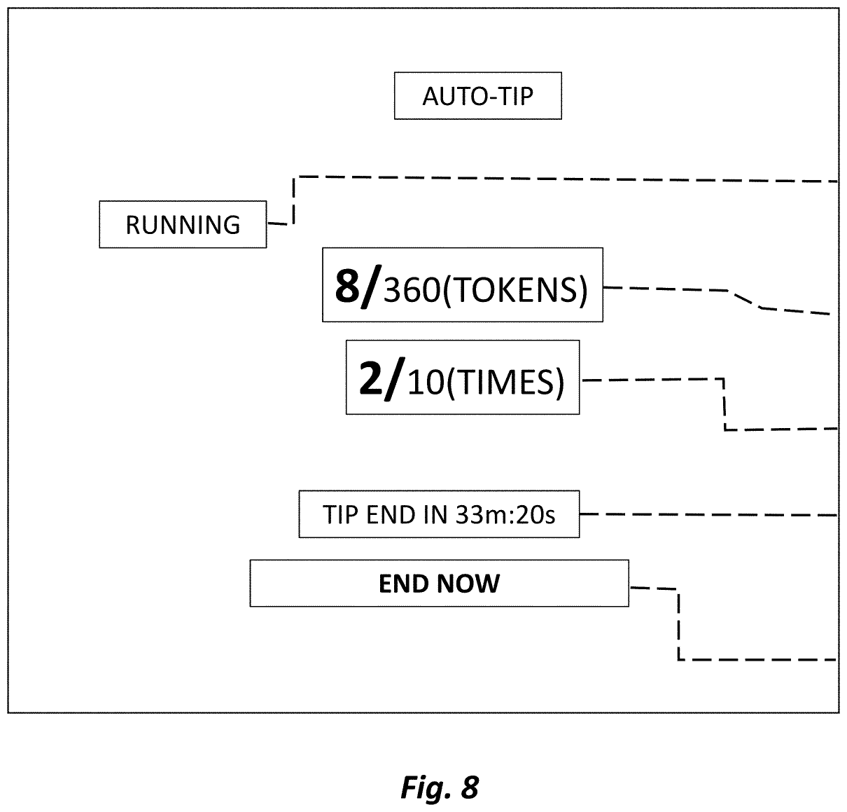

FIGS. 7 and 8 illustrate an exemplary disclosed tip operation that may be an automatic tipping operation. Users may utilize the exemplary disclosed GUI to set a plurality of graphical elements (e.g., a graphical element 352, a graphical element 354, and/or a graphical element 356). A user may set an amount of a tip to be provided via setting graphical element 352. A user may set a number of times a tip is to be provided via setting graphical element 354. A user may set an interval at which a tip may be provided via setting graphical element 356. A user may thereby preset automatic tipping to be provided automatically (e.g., a user may initiate a desired present tipping pattern via the exemplary disclosed GUI), including a preset amount, number of times, and/or interval. In at least some exemplary embodiments, a user (e.g., user 320) may enter input to the exemplary disclosed GUI (e.g., touch a preset graphical button) to initiate a desired automatic tipping pattern. A user may thereby execute (e.g., start and stop) a desired tipping operation when desired. For example, a preset automatic tipping operation may be stopped and/or stopped based on a single step (e.g., pressing a graphical element on the exemplary disclosed GUI).

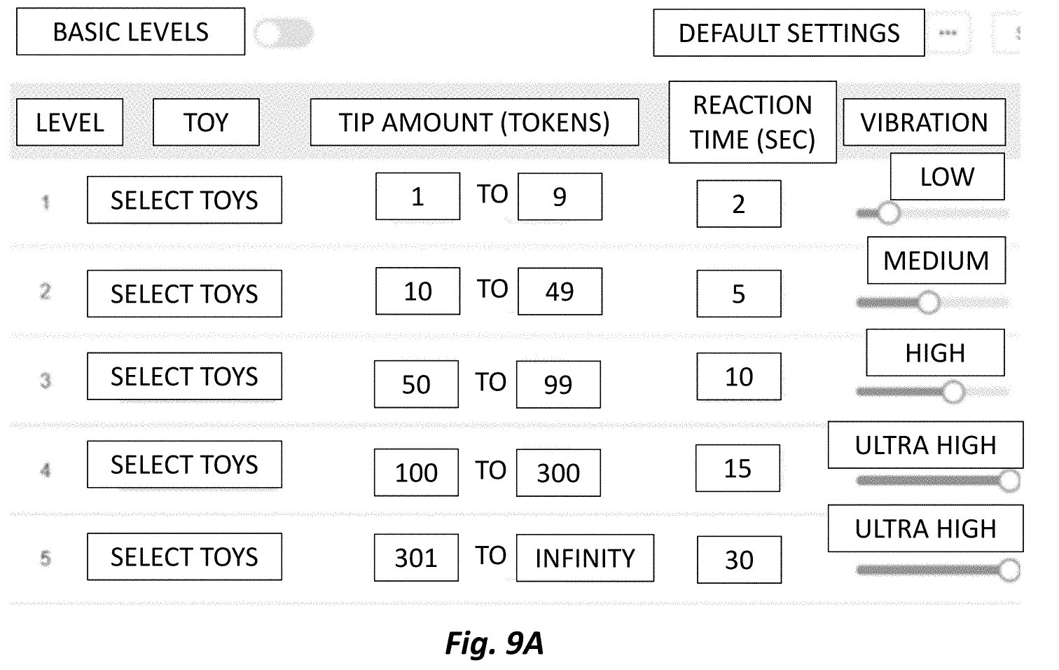

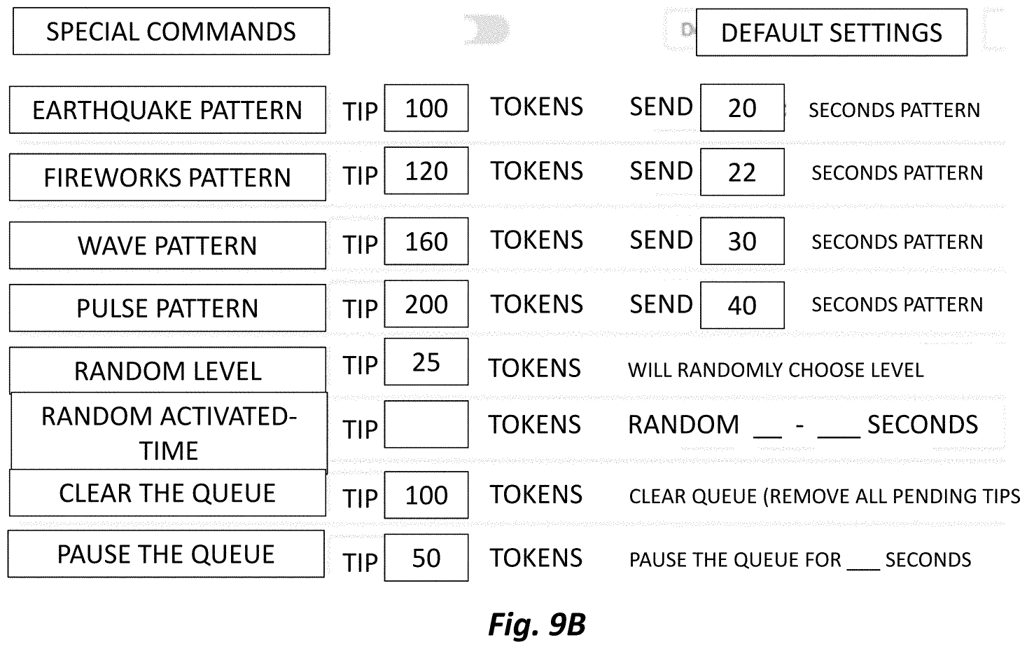

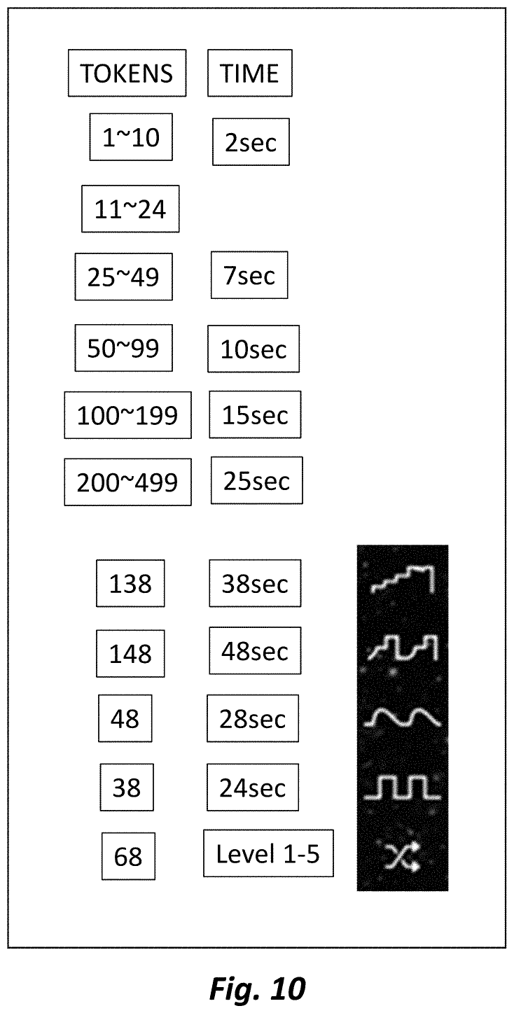

FIGS. 9A and 9B illustrate an exemplary disclosed settings display of the exemplary disclosed GUI. For example, a plurality of tipping rules may be displayed via the exemplary disclosed GUI. The rules may be set by user 320, model 325, system 300 (e.g., via an operation of the exemplary disclosed module including algorithms and/or artificial intelligence operations), and/or any other suitable criteria. For example, settings for certain toys (e.g., viewer accessory 308 and/or model accessory 315) may be provided. A level of operation may be provided based on tipping ranges. A reaction time and/or vibration level may also be set. Desired patterns of toy operation may also be set. Any desired operation, level, and/or any other desired criteria may be set based on a desired amount and/or range of tipping. As illustrated in FIG. 10, the exemplary disclosed tipping rules described above may be displayed via the exemplary disclosed GUI (e.g., tipping rules of the settings such as model settings may be displayed on the exemplary disclosed live broadcast room page). For example, FIG. 10 may include tipping information similar to for example as described above regarding graphical element 344.

As illustrated in the various exemplary embodiments of FIGS. 4-10, system 300 may operate to provide any suitable tipping operations. After a user activates a graphical element via the exemplary disclosed GUI (e.g., presses a graphical button), system 300 may operate to provide one or more tip operations. For example, at least one tip operation may be performed on the exemplary disclosed live broadcast platform associated with the tip information (e.g., as described above). For example, a tip operation (e.g., quick-tip or auto-tip for example as described herein) may be performed in real-time or near real-time with a broadcast (e.g., performance displayed on the exemplary disclosed live broadcast platform). Also for example, a tip operation (e.g., quick-tip or auto-tip for example as described herein) may be performed at a desired time (e.g., a predetermined time) during a broadcast (e.g., performance displayed on the exemplary disclosed live broadcast platform). Further for example, a plurality of tip operations (e.g., two or more tip operations for example as described herein) may be performed within a predetermined time period during a broadcast (e.g., displayed on the exemplary disclosed live broadcast platform). The exemplary disclosed tipping items displayed on the GUI may include one or more icons, one or more module elements, one or more bullet screens, and/or any other desired elements. The exemplary disclosed tip operation may include tipping virtual currency of a specified value, tipping a virtual gift, tipping a specified message, and/or any other desired item of value.

As illustrated in the various exemplary embodiments of FIGS. 4-10, system 300 may operate to provide any suitable tipping operations such as, for example, quick-tip and/or auto-tip operations. For example, a viewer may access a live broadcast platform website through an application integrated in a specified browser on a device (e.g., user device 305 or model device 310). The live broadcasting platform may include at least one multimedia content page. After entering a live broadcast room page, the viewer (e.g., user 305) may watch a performance and send tips to a model (e.g., model 325) of the live broadcast room. One or more tipping items may be output (e.g., displayed via the exemplary disclosed GUI) as icons and/or module elements on the multimedia content page (e.g., as illustrated in FIGS. 4-10). After system 300 operates to execute a tip operation, adult toys (e.g., one or more viewer accessories 308 and/or model accessories 315) connected with one or more viewer devices and/or model devices (e.g., one or more user devices 305 and/or model devices 310) may perform a predefined action including a predefined action type and action intensity (e.g., vibration, a suction operation, rotation, swinging, contracting, expanding, performing an inhalation action, heating, cooling, undergoing temperature variation, and/or any other suitable action or function).

In at least some exemplary embodiments, system 300 may provide a quick-tip operation. A trigger instruction associated with one or more tipping items may be generated when a viewer (e.g., user 320) selects a tipping item (e.g., the exemplary disclosed graphical elements). Responsive to the trigger instruction of one or more tipping items, system 300 may execute one or more tip operations during a broadcast of the broadcast platform. The user may thereby conveniently tip in real-time or near real-time during a live broadcast via the GUI.

In at least some exemplary embodiments, system 300 may provide an auto-tip operation. A trigger instruction associated with one or more tipping items may be generated when a viewer (e.g., user 320) selects a tipping item (e.g., an exemplary disclosed graphical element), which may include at least one item of data of the tipping items. Responsive to the trigger instruction of the tipping items, system 300 may execute a tip operation during a broadcast of the broadcast platform based on the tipping items (e.g., correlative to the tipping items) after any suitable time intervals and/or other suitable criteria (e.g., for example as described above regarding FIGS. 7 and 8).

In at least some exemplary embodiments, system 300 may execute at least one tip operation on one or more live broadcast platforms for example as described above regarding FIGS. 4-10. The at least one tip operation may be associated with one or more tipping items that may include the exemplary disclosed tipping graphical elements (e.g., graphical elements 340, 342, 346, 348, 350, 352, 354, and/or 356) and may be responsive to a trigger instruction of any of the one or more tipping graphical elements. The trigger instruction may include one or more combinations of touch trigger instruction, button trigger instruction, sound trigger instruction, operation trigger instruction, gesture trigger instruction, and/or human biometric recognition trigger instruction. System 300 may execute at least one tip operation on the exemplary disclosed live broadcast platform correlating to tipping items in response to a trigger instruction of any of said tipping items, including executing at least one tipping operation on the live broadcast platform correlating to the tipping items according to a preset script and/or a preset interface associated with the live broadcasting platform (e.g., that may be responsive to a trigger instruction of any of the tipping items). Before displaying one or more multimedia content pages on the user interface of the exemplary disclosed viewer devices, system 300 (e.g., including the exemplary disclosed software application) may determine whether there is a preset script or a preset interface corresponding to the live broadcast platform correlating to the multimedia content page (e.g., if there is, system 300 may display one or more exemplary disclosed tipping graphical elements on the user interface of the viewer devices). Different live broadcast platforms of system 300 may be configured to have a different preset script or preset interface. The tip operation may include tipping specified amounts of virtual currency, tipping a specified virtual gift, and/or tipping specified information. Displaying one or more multimedia content pages on the user interface of the viewer devices may include displaying an interactive panel on the user interface of the viewer devices for example as illustrated in FIGS. 4-10, wherein one or more tipping items may be configured to display on the interactive panel. Each element (e.g., piece) of the tipping items may be configured with a control layout on the interactive panel, and system 300 may execute at least one tip operation on the live broadcast platform correlating to tipping items responsive to a trigger instruction of any of said tipping items. System 300 may generate a trigger instruction of the tipping items responsive to a selective operation of the control of any of tipping items (e.g., via the exemplary disclosed user device and GUI). System 300 may execute at least one tip operation on the live broadcast platform correlating to the tipping items in response to the trigger instruction of the tipping items.

In at least some exemplary embodiments and for example as illustrated in FIGS. 5-10, one or more tipping items may include one or more different fixed values that may include a preset fixed value or a customized value set by the exemplary disclosed user devices (e.g., viewer device such as user devices 305 and/or model devices 310) and/or or one or more ranges of value. A target value may be selected inside or outside of the range of values, and/or a target value may be set from the range of values randomly via operation of the exemplary disclosed user device (e.g., viewer device). Tipping items may include at least one item of data for a target number of tip operations, an interval time of each tip operation, a total time of a tip operation, a resource and/or criteria of each tip operation, and/or a total resource of the tip operation. Each item of data may be set through the exemplary disclosed user device (e.g., viewer device) or according to a preset algorithm. System 300 (e.g., and the exemplary disclosed software application) may be configured to, responsive to the tip operation, send a control signal to the exemplary disclosed adult toys to drive the adult toys to execute a predefined action for example as described herein. System 300 may include at least two adult toys (e.g., viewer accessory 308 and/or model accessory 315) configured to communicate with the user device (e.g., viewer device) correlating to the multimedia content pages. The software application may be configured to, responsive to the tip operation from any of the viewer devices, send a control signal to the adult toys to drive the adult toys to execute a predefined action. System 300 may include an adult toy (e.g., model accessory 315) and a model device (e.g., model device 310). The adult toy may be configured to communicate with the model device and receive signals. The model device may be configured to communicate with the broadcast platform and receive tips. Responsive to the received tip, the model device may send a control signal to the adult toy to drive the adult toy to execute a predefined action. The predefined action may include any suitable predefined action type and/or predefined action level. For example, the predefined action type may comprise one or a combination of the following: vibration, rotation, suction, scaling, bending, temperature control, expansion, and/or reciprocating movement. Also for example, the predefined action level may comprise one or a combination of the following: amplitude, frequency, acceleration, temperature, reciprocating times, and/or duration. The predefined action may comprise a control pattern, the control pattern including a set of instructions to execute the action type and action level based on time order. The tipping items may be received from the live broadcast platform, including receiving from a tip rule correlating to a multimedia content page of the live broadcast platform, and/or receiving from the multimedia content page.

An exemplary operation of the exemplary disclosed system, apparatus, and method will now be described. FIG. 4 illustrates an exemplary process 400 of system 300. Process 400 begins at step 405.

At step 410, system 300 may be configured, re-configured, and/or communication provided by system 300 may be maintained. For example, system 300 may be configured as illustrated in FIGS. 1 and 2 or with any other suitable configuration. System 300 may provide a communication interface such as a chat room or interactive application to one or more users 320 and/or one or more models 325 such as, for example, as illustrated in FIGS. 3-10. One or more users 320 and one or more models 325 may thereby communicate and interact with each other using live video communication in real-time or near real-time. Any desired number and arrangement of user devices 305, viewer accessories 308, model devices 310, and model accessories 315 may be included in the configuration of system 300. One or more viewer accessories 308 and/or one or more model accessories 315 (e.g., sexual stimulation devices such as operable adult toys) may be configured to receive data and signals from other components of system 300 for example as described herein. The exemplary disclosed module, storage (e.g., storage buffer), and hardware may include a memory having stored thereon instructions, a processor configured to execute the instructions resulting in a software application, and a software application configured to perform process 400.

In at least some exemplary embodiments at step 410, user 320 may install an application of system 300 on user device 305 (e.g., and/or model 325 may install the application on model device 310). User 320 (e.g., and/or model 325) may authorize the application to access and control functions of user device 305, viewer accessory 308, model device 310, and/or model accessory 315. User 320 (e.g., and/or model 325) may also authorize the application to access the data of Bluetooth or any other suitable communication components of user device 305 (e.g., and/or model device 310). Model 325 (e.g., and/or user 320) may also connect model accessory 315 (e.g., and/or viewer accessory 308) to model device 310 (e.g., and/or user device 305) via Bluetooth or any other suitable communication technique. In at least some exemplary embodiments, viewer accessory 308 and/or model accessory 315 may be connected by the exemplary disclosed short range communication techniques to user device 305 and/or model device 310 and operated by user 320 and/or model 325 (e.g., user 320 and/or model 325 may control any of the exemplary disclosed devices directly or via network 330).

At step 415, system 300 may configure tipping information for example as described above regarding FIGS. 5-10. Tipping information may be configured based on input provided by a user (e.g., one or more users 320 and/or models 325) via the exemplary disclosed GUI for example as described herein, predetermined and/or set by system 300 based on an algorithm or machine learning (e.g., based on past usage of the user), and/or any other suitable criteria.

At step 420, system 300 may provide data to be displayed to a user such as, for example, live broadcast data (e.g., via the exemplary disclosed live broadcast platform website). For example, any desired performance (e.g., adult entertainment) may be provided (e.g., broadcast and displayed) via the exemplary disclosed application and user devices (e.g., via the exemplary disclosed GUI using one or more user devices 305 and/or model devices 310).

At step 425, system 300 may perform tipping operations. For example as described above regarding FIGS. 4-10, one or more users may utilize the exemplary disclosed GUI to perform tipping operations. For example, a viewer such as a user 320 may use system 300 to perform the exemplary disclosed quick-tip or auto-tip operations.

At step 430, system 300 may determine whether or not tipping information should be reconfigured. System 300 may make this determination based on user input (e.g., of one or more users 320 and/or models 325), predetermined algorithms, a time duration being reached, artificial intelligence operations, and/or any other suitable criteria. If system 300 determines that tipping information is to be reconfigured, system 300 may return to step 415. System 300 may repeat steps 415 through 430 for any desired number of iterations. If tipping information is not to be reconfigured, system 300 may proceed to step 435.

At step 435, system 300 may determine whether or not system 300 is to be reconfigured based on, for example, input provided by user 320 and/or model 325, a predetermined operation or algorithm of the exemplary disclosed module, and/or any other suitable criteria. If system 300 is to be reconfigured, system 300 may return to step 410. System 300 may repeat steps 410 through 435 for any desired number of iterations. If system 300 is not to be reconfigured, system 300 may proceed to step 440, at which process 400 ends.

In at least some exemplary embodiments, the exemplary disclosed system method may include a viewer device, a memory having stored thereon instructions, and a processor to execute the instructions resulting in a software application. The software application may be configured to: display one or more multimedia content pages on user interface of the viewer devices, the one or more multimedia content pages and the one or more live broadcast platforms associated with (e.g., correlating to) each other; display one or more tipping items on user interfaces of the viewer devices, one or more tipping items and at least one multimedia content page correlating to each other; and execute at least one tip operation on a live broadcast platform correlating to the tipping items responsive to a trigger instruction of any of the tipping items.

In at least some exemplary embodiments, the exemplary disclosed system may include a tipping module, comprising computer-executable code stored in non-volatile memory, a processor, and a user device (e.g., user device 305 or model device 310). The tipping module, the processor, and the user device may be configured to display one or more multimedia content pages on a user interface of the user device, the one or more multimedia content pages associated with one or more live broadcast platforms, display one or more tipping graphical elements on the user interface of the user device, the one or more tipping graphical elements associated with the one or more multimedia content pages, and execute at least one tip operation on the one or more live broadcast platforms, the at least one tip operation associated with the one or more tipping graphical elements and responsive to a trigger instruction of any of the one or more tipping graphical elements. The trigger instruction may include at least one selected from the group of a touch trigger instruction, a button trigger instruction, a sound trigger instruction, an operation trigger instruction, a gesture trigger instruction, a human biometric recognition trigger instruction, and combinations thereof. Executing the at least one tip operation may include executing the at least one tip operation on the one or more live broadcast platforms associated with the one or more tipping graphical elements according to at least one of a preset script or a preset interface. The tipping module, the processor, and the user device may be configured to, before displaying the one or more tipping graphical elements on the user interface, determine whether the preset script or the preset interface is associated with the one or more live broadcast platforms, and if the preset script or the preset interface is associated with the one or more live broadcast platforms, display the one or more tipping graphical elements on the user interface. Different live broadcast platforms of the one or more live broadcast platforms may be configured to have different preset scripts or preset interfaces. The at least one tip operation may include at least one selected from the group of tipping a specified amount of virtual currency, tipping a specified virtual gift, tipping specified information, and combinations thereof. Displaying the one or more tipping graphical elements on the user interface may include displaying an interactive panel on the user interface, wherein the one or more tipping graphical elements may be configured to display on the interactive panel. Each of the one or more tipping graphical elements may be configured with a control layout on the interactive panel. Executing the at least one tip operation on the one or more live broadcast platforms may include generating the trigger instruction of the one or more tipping graphical elements responsive to a selective operation of control of any of the one or more tipping graphical elements. Executing the at least one tip operation on the one or more live broadcast platforms may be associated with the one or more tipping graphical elements in response to the trigger instruction. The one or more tipping graphical elements may include at least one selected from the group of one or more different fixed values that include a preset fixed value or a customized value set by the user device, one or more ranges of values, and combinations thereof. The tipping module, the processor, and the user device may be configured to select a target value inside or outside of the one or more ranges of values, or to select the target value from the one or more ranges of values randomly by using the user device. The one or more tipping graphical elements may include at least one selected from the group of a target number of the at least one tip operation, an interval time of the at least one tip operation, a total time of the at least one tip operation, a resource of the at least one tip operation, a total resource of the at least one tip operation, and combinations thereof. Data of the one or more tipping graphical elements may be set via the user device or based on a preset algorithm. The exemplary disclosed system may also include at least one adult toy configured to communicate with the user device and receive a signal. The tipping module, the processor, and the user device may be configured to, responsive to the at least one tip operation, send the signal to the at least one adult toy to drive the at least one adult toy to execute a predefined action. The exemplary disclosed system may further include at least two adult toys configured to communicate with the user device. The tipping module, the processor, and the user device may be configured to, responsive to the at least one tip operation from the user device, send a control signal to the at least two adult toys to drive the at least two adult toys to execute a predefined action. The exemplary disclosed system may also include an adult toy and a model device. The adult toy may be configured to communicate with the model device and receive a signal. The model device may be configured to communicate with the one or more live broadcast platforms and receive a tip based on the at least one tip operation. Responsive to the received tip, the model device may be configured to send the signal to the adult toy to drive the adult toy to execute a predefined action. The predefined action may be executed based on a predefined action type and a predefined action level. The predefined action type may include at least one selected from the group of vibration, rotation, suction, scaling, bending, temperature control, expansion, reciprocating movement, and combinations thereof. The predefined action level may include at least one selected from the group of amplitude, frequency, acceleration, temperature, reciprocating times, duration, and combinations thereof. The predefined action may be executed based on a control pattern, the control pattern including a set of instructions to execute the action type and the action level based on time order. The one or more tipping graphical elements may be received from the one or more live broadcast platforms, the receiving including receiving a tip rule correlating to the one or more multimedia content pages or receiving data from the one or more multimedia content pages.

In at least some exemplary embodiments, the exemplary disclosed system method may include displaying one or more multimedia content pages on a user interface of a user device, the one or more multimedia content pages associated with one or more live broadcast platforms, displaying one or more tipping graphical elements on the user interface of the user device, the one or more tipping graphical elements associated with the one or more multimedia content pages, and executing at least one tip operation on the one or more live broadcast platforms, the at least one tip operation associated with the one or more tipping graphical elements and responsive to a trigger instruction of any of the one or more tipping graphical elements. The exemplary disclosed method may also include, before displaying the one or more tipping graphical elements on the user interface, determining whether a preset script or a preset interface is associated with the one or more live broadcast platforms. The exemplary disclosed method may further include, if the preset script or the preset interface is associated with the one or more live broadcast platforms, displaying the one or more multimedia content pages on the user interface.

In at least some exemplary embodiments, the exemplary disclosed system may include a user device having a user interface, a model device, one or more live broadcast platforms, and at least one adult toy. The user device may be configured to display one or more multimedia content pages on the user interface, the one or more multimedia content pages associated with one or more live broadcast platforms. The user device may be configured to display one or more tipping graphical elements on the user interface, the one or more tipping graphical elements associated with the one or more multimedia content pages. At least one tip operation may be executed on the one or more live broadcast platforms, the at least one tip operation associated with the one or more tipping graphical elements and responsive to a trigger instruction of any of the one or more tipping graphical elements. The model device may be configured to receive a tip based on the at least one tip operation. The model device may be configured to send a control signal, responsive to the received tip, to the at least one adult toy to drive the at least one adult toy to execute a predefined action. The predefined action may include at least one selected from the group of vibration, rotation, suction, scaling, bending, temperature control, expansion, reciprocating movement, and combinations thereof.

The exemplary disclosed system, apparatus, and method may provide an efficient and effective technique for providing a tip during a live broadcast. For example, the exemplary disclosed system, apparatus, and method may provide a technique for a user to conveniently provide a tip during a live broadcast without being distracted from the live broadcast. The exemplary disclosed system, apparatus, and method may also increase an amount of tipping made during a live broadcast by providing an efficient and easy technique for tipping. For example, the exemplary disclosed system, apparatus, and method may allow for viewers to conveniently send tips, and may also simplify a process for sending tips. The exemplary disclosed system, apparatus, and method may encourage tipping by users during a live broadcast (e.g., based on convenient tipping) and increase interaction between models and viewer.

In at least some exemplary embodiments, the exemplary disclosed system, apparatus, and method may utilize sophisticated machine learning and/or artificial intelligence techniques to prepare and submit datasets and variables to cloud computing clusters and/or other analytical tools (e.g., predictive analytical tools) which may analyze such data using artificial intelligence neural networks. The exemplary disclosed system may for example include cloud computing clusters performing predictive analysis. For example, the exemplary neural network may include a plurality of input nodes that may be interconnected and/or networked with a plurality of additional and/or other processing nodes to determine a predicted result. Exemplary artificial intelligence processes may include filtering and processing datasets, processing to simplify datasets by statistically eliminating irrelevant, invariant or superfluous variables or creating new variables which are an amalgamation of a set of underlying variables, and/or processing for splitting datasets into train, test and validate datasets using at least a stratified sampling technique. The exemplary disclosed system may utilize prediction algorithms and approach that may include regression models, tree-based approaches, logistic regression, Bayesian methods, deep-learning and neural networks both as a stand-alone and on an ensemble basis, and final prediction may be based on the model/structure which delivers the highest degree of accuracy and stability as judged by implementation against the test and validate datasets.

An illustrative representation of a computing device appropriate for use with embodiments of the system of the present disclosure is shown in FIG. 12. The computing device 100 can generally be comprised of a Central Processing Unit (CPU, 101), optional further processing units including a graphics processing unit (GPU), a Random Access Memory (RAM, 102), a mother board 103, or alternatively/additionally a storage medium (e.g., hard disk drive, solid state drive, flash memory, cloud storage), an operating system (OS, 104), one or more application software 105, a display element 106, and one or more input/output devices/means 107, including one or more communication interfaces (e.g., RS232, Ethernet, Wifi, Bluetooth, USB). Useful examples include, but are not limited to, personal computers, smart phones, laptops, mobile computing devices, tablet PCs, touch boards, and servers. Multiple computing devices can be operably linked to form a computer network in a manner as to distribute and share one or more resources, such as clustered computing devices and server banks/farms.

Various examples of such general-purpose multi-unit computer networks suitable for embodiments of the disclosure, their typical configuration and many standardized communication links are well known to one skilled in the art, as explained in more detail and illustrated by FIG. 13, which is discussed herein-below.

According to an exemplary embodiment of the present disclosure, data may be transferred to the system, stored by the system and/or transferred by the system to users of the system across local area networks (LANs) (e.g., office networks, home networks) or wide area networks (WANs) (e.g., the Internet). In accordance with the previous embodiment, the system may be comprised of numerous servers communicatively connected across one or more LANs and/or WANs. One of ordinary skill in the art would appreciate that there are numerous manners in which the system could be configured and embodiments of the present disclosure are contemplated for use with any configuration.

In general, the system and methods provided herein may be employed by a user of a computing device whether connected to a network or not. Similarly, some steps of the methods provided herein may be performed by components and modules of the system whether connected or not. While such components/modules are offline, and the data they generated will then be transmitted to the relevant other parts of the system once the offline component/module comes again online with the rest of the network (or a relevant part thereof). According to an embodiment of the present disclosure, some of the applications of the present disclosure may not be accessible when not connected to a network, however a user or a module/component of the system itself may be able to compose data offline from the remainder of the system that will be consumed by the system or its other components when the user/offline system component or module is later connected to the system network.

Referring to FIG. 13, a schematic overview of a system in accordance with an embodiment of the present disclosure is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to an exemplary embodiment, as shown in FIG. 13, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present disclosure may or may not utilize one or more routers 202. One of ordinary skill in the art would appreciate that there are numerous ways server 203 may connect to WAN 201 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 203 via WAN 201 or other network in numerous ways. For instance, a component or module may connect to the system i) through a computing device 212 directly connected to the WAN 201, ii) through a computing device 205, 206 connected to the WAN 201 through a routing device 204, iii) through a computing device 208, 209, 210 connected to a wireless access point 207 or iv) through a computing device 211 via a wireless connection (e.g., CDMA, GMS, 3G, 4G) to the WAN 201. One of ordinary skill in the art will appreciate that there are numerous ways that a component or module may connect to server 203 via WAN 201 or other network, and embodiments of the present disclosure are contemplated for use with any method for connecting to server 203 via WAN 201 or other network. Furthermore, server 203 could be comprised of a personal computing device, such as a smartphone, acting as a host for other computing devices to connect to.

The communications means of the system may be any means for communicating data, including image and video, over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

Traditionally, a computer program includes a finite sequence of computational instructions or program instructions. It will be appreciated that a programmable apparatus or computing device can receive such a computer program and, by processing the computational instructions thereof, produce a technical effect.

A programmable apparatus or computing device includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computing device can include any and all suitable combinations of at least one general purpose computer, special-purpose computer, programmable data processing apparatus, processor, processor architecture, and so on. It will be understood that a computing device can include a computer-readable storage medium and that this medium may be internal or external, removable and replaceable, or fixed. It will also be understood that a computing device can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the disclosure as claimed herein could include an optical computer, quantum computer, analog computer, or the like.

Regardless of the type of computer program or computing device involved, a computer program can be loaded onto a computing device to produce a particular machine that can perform any and all of the depicted functions. This particular machine (or networked configuration thereof) provides a technique for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Illustrative examples of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A data store may be comprised of one or more of a database, file storage system, relational data storage system or any other data system or structure configured to store data. The data store may be a relational database, working in conjunction with a relational database management system (RDBMS) for receiving, processing and storing data. A data store may comprise one or more databases for storing information related to the processing of moving information and estimate information as well one or more databases configured for storage and retrieval of moving information and estimate information.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software components or modules, or as components or modules that employ external routines, code, services, and so forth, or any combination of these. All such implementations are within the scope of the present disclosure. In view of the foregoing, it will be appreciated that elements of the block diagrams and flowchart illustrations support combinations of means for performing the specified functions, combinations of steps for performing the specified functions, program instruction technique for performing the specified functions, and so on.

It will be appreciated that computer program instructions may include computer executable code. A variety of languages for expressing computer program instructions are possible, including without limitation C, C++, Java, JavaScript, assembly language, Lisp, HTML, Perl, and so on. Such languages may include assembly languages, hardware description languages, database programming languages, functional programming languages, imperative programming languages, and so on. In some embodiments, computer program instructions can be stored, compiled, or interpreted to run on a computing device, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computing device enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computing device can process these threads based on priority or any other order based on instructions provided in the program code.

Unless explicitly stated or otherwise clear from the context, the verbs “process” and “execute” are used interchangeably to indicate execute, process, interpret, compile, assemble, link, load, any and all combinations of the foregoing, or the like. Therefore, embodiments that process computer program instructions, computer-executable code, or the like can suitably act upon the instructions or code in any and all of the ways just described.

The functions and operations presented herein are not inherently related to any particular computing device or other apparatus. Various general-purpose systems may also be used with programs in accordance with the teachings herein, or it may prove convenient to construct more specialized apparatus to perform the required method steps. The required structure for a variety of these systems will be apparent to those of ordinary skill in the art, along with equivalent variations. In addition, embodiments of the disclosure are not described with reference to any particular programming language. It is appreciated that a variety of programming languages may be used to implement the present teachings as described herein, and any references to specific languages are provided for disclosure of enablement and best mode of embodiments of the disclosure. Embodiments of the disclosure are well suited to a wide variety of computer network systems over numerous topologies. Within this field, the configuration and management of large networks include storage devices and computing devices that are communicatively coupled to dissimilar computing and storage devices over a network, such as the Internet, also referred to as “web” or “world wide web”.

Throughout this disclosure and elsewhere, block diagrams and flowchart illustrations depict methods, apparatuses (e.g., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “component”, “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present disclosure are contemplated for use with any language.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

It will be apparent to those skilled in the art that various modifications and variations can be made to the disclosed system and method. Other embodiments will be apparent to those skilled in the art from consideration of the specification and practice of the disclosed method and apparatus. It is intended that the specification and examples be considered as exemplary only, with a true scope being indicated by the following claims.

What is claimed is:

1. A system, comprising:
a tipping module, comprising computer-executable code stored in non-volatile memory;
a processor; and
a user device;
wherein the tipping module, the processor, and the user device are configured to:
display one or more multimedia content pages on a user interface of the user device, the one or more multimedia content pages associated with one or more live broadcast platforms;
display one or more tipping graphical elements on the user interface of the user device, the one or more tipping graphical elements associated with the one or more multimedia content pages; and
execute at least one tip operation on the one or more live broadcast platforms, the at least one tip operation associated with the one or more tipping graphical elements and responsive to a trigger instruction of any of the one or more tipping graphical elements.

2. The system of claim 1, wherein the trigger instruction includes at least one selected from the group of a touch trigger instruction, a button trigger instruction, a sound trigger instruction, an operation trigger instruction, a gesture trigger instruction, a human biometric recognition trigger instruction, and combinations thereof.

3. The system of claim 1, wherein executing the at least one tip operation includes executing the at least one tip operation on the one or more live broadcast platforms associated with the one or more tipping graphical elements according to at least one of a preset script or a preset interface.

4. The system of claim 3, wherein:
the tipping module, the processor, and the user device are configured to, before displaying the one or more tipping graphical elements on the user interface, determine whether the preset script or the preset interface is associated with the one or more live broadcast platforms; and
if the preset script or the preset interface is associated with the one or more live broadcast platforms, display the one or more tipping graphical elements on the user interface.

5. The system of claim 4, wherein different live broadcast platforms of the one or more live broadcast platforms are configured to have different preset scripts or preset interfaces.

6. The system of claim 1, wherein the at least one tip operation includes at least one selected from the group of tipping a specified amount of virtual currency, tipping a specified virtual gift, tipping specified information, and combinations thereof.

7. The system of claim 1, wherein displaying the one or more tipping graphical elements on the user interface includes displaying an interactive panel on the user interface, wherein the one or more tipping graphical elements are configured to display on the interactive panel.

8. The system of claim 7, wherein:
each of the one or more tipping graphical elements is configured with a control layout on the interactive panel;
executing the at least one tip operation on the one or more live broadcast platforms includes generating the trigger instruction of the one or more tipping graphical elements responsive to a selective operation of control of any of the one or more tipping graphical elements; and
executing the at least one tip operation on the one or more live broadcast platforms is associated with the one or more tipping graphical elements in response to the trigger instruction.

9. The system of claim 1, wherein:
the one or more tipping graphical elements include at least one selected from the group of one or more different fixed values that include a preset fixed value or a customized value set by the user device, one or more ranges of values, and combinations thereof; and
the tipping module, the processor, and the user device are configured to select a target value inside or outside of the one or more ranges of values, or to select the target value from the one or more ranges of values randomly by using the user device.

10. The system of claim 1, wherein:
the one or more tipping graphical elements include at least one selected from the group of a target number of the at least one tip operation, an interval time of the at least one tip operation, a total time of the at least one tip operation, a resource of the at least one tip operation, a total resource of the at least one tip operation, and combinations thereof; and
data of the one or more tipping graphical elements is set via the user device or based on a preset algorithm.

11. The system of claim 1, further comprising at least one adult toy configured to communicate with the user device and receive a signal;
wherein the tipping module, the processor, and the user device are configured to, responsive to the at least one tip operation, send the signal to the at least one adult toy to drive the at least one adult toy to execute a predefined action.

12. The system of claim 1, further comprising at least two adult toys configured to communicate with the user device;
wherein the tipping module, the processor, and the user device are configured to, responsive to the at least one tip operation from the user device, send a control signal to the at least two adult toys to drive the at least two adult toys to execute a predefined action.

13. The system of claim 1, further comprising an adult toy and a model device; wherein:
the adult toy is configured to communicate with the model device and receive a signal;
the model device is configured to communicate with the one or more live broadcast platforms and receive a tip based on the at least one tip operation; and
responsive to the received tip, the model device is configured to send the signal to the adult toy to drive the adult toy to execute a predefined action.

14. The system of claim 13, wherein:
the predefined action is executed based on a predefined action type and a predefined action level;
the predefined action type includes at least one selected from the group of vibration, rotation, suction, scaling, bending, temperature control, expansion, reciprocating movement, and combinations thereof; and
the predefined action level includes at least one selected from the group of amplitude, frequency, acceleration, temperature, reciprocating times, duration, and combinations thereof.

15. The system of claim 14, wherein the predefined action is executed based on a control pattern, the control pattern including a set of instructions to execute the action type and the action level based on time order.

16. The system of claim 1, wherein the one or more tipping graphical elements is received from the one or more live broadcast platforms, the receiving including receiving a tip rule correlating to the one or more multimedia content pages or receiving data from the one or more multimedia content pages.

17. A method, comprising:
displaying one or more multimedia content pages on a user interface of a user device, the one or more multimedia content pages associated with one or more live broadcast platforms;
displaying one or more tipping graphical elements on the user interface of the user device, the one or more tipping graphical elements associated with the one or more multimedia content pages; and
executing at least one tip operation on the one or more live broadcast platforms, the at least one tip operation associated with the one or more tipping graphical elements and responsive to a trigger instruction of any of the one or more tipping graphical elements.

18. The method of claim 17, further comprising:
before displaying the one or more tipping graphical elements on the user interface, determining whether a preset script or a preset interface is associated with the one or more live broadcast platforms; and
if the preset script or the preset interface is associated with the one or more live broadcast platforms, displaying the one or more multimedia content pages on the user interface.

19. A system, comprising:
a user device having a user interface;
a model device;
one or more live broadcast platforms; and
at least one adult toy;
wherein the user device is configured to display one or more multimedia content pages on the user interface, the one or more multimedia content pages associated with one or more live broadcast platforms;
wherein the user device is configured to display one or more tipping graphical elements on the user interface, the one or more tipping graphical elements associated with the one or more multimedia content pages;
wherein at least one tip operation is executed on the one or more live broadcast platforms, the at least one tip operation associated with the one or more tipping graphical elements and responsive to a trigger instruction of any of the one or more tipping graphical elements;
wherein the model device is configured to receive a tip based on the at least one tip operation; and
wherein the model device is configured to send a control signal, responsive to the received tip, to the at least one adult toy to drive the at least one adult toy to execute a predefined action.

20. The system of claim 19, wherein the predefined action includes at least one selected from the group of vibration, rotation, suction, scaling, bending, temperature control, expansion, reciprocating movement, and combinations thereof.
